(19) United States
(12) Patent Application Publication
(10) Pub. No.: US 2013/0071938 A1
(43) Pub. Date: Mar. 21, 2013

(54) METHOD FOR ANALYZING SUGAR CHAIN BY MASS SPECTROMETRY

(71) Applicants: Hideyuki Shimaoka, Shinagawa-ku (JP); Midori Abe, Shinagawa-ku (JP); Kohta Igarashi, Shinagawa-ku (JP)

(72) Inventors: Hideyuki Shimaoka, Shinagawa-ku (JP); Midori Abe, Shinagawa-ku (JP); Kohta Igarashi, Shinagawa-ku (JP)

(73) Assignee: SUMITOMO BAKELITE CO., LTD., Shinagawa-ku (JP)

(21) Appl. No.: 13/634,683

(22) PCT Filed: Mar. 17, 2011
(86) PCT No.: PCT/JP2011/056405

§ 371 Date: Nov. 28, 2012

(30) Foreign Application Priority Data
Mar. 18, 2010 (JP) 2010062174

(51) Int. Cl. G01N 33/50

(52) U.S. Cl. CPC G01N 33/50
USPC 436/94

(57) ABSTRACT

It has been found out that it is possible to increase the ionization efficiency of a sample sugar chain by methylating hydroxyl groups of the sugar chain before MALDI-TOF MS measurement. This enables quantitative and structural analyses on the sample sugar chain with high accuracy.

### TECHNICAL FIELD

The present invention relates to a method for analyzing a sugar chain contained in a biological sample by mass spectrometry.

### BACKGROUND ART

Sugar chain is a general term for molecules composed of monosaccharides such as glucose, galactose, mannose, fucose, xylose, N-acetylglucosamine, N-acetylgalactosamine, and sialic acid, and derivatives thereof, linked via a glycosidic bond to give a chain form.

There are a great diversity of sugar chains, which are substances taking part in various functions possessed by naturally-occurring organisms. Sugar chain often exists in vivo by binding to a protein, a lipid, or the like in the form of glycoconjugate, and is one of important constituents of bodies. It is about to be revealed that sugar chain in vivo is closely associated with intercellular communications, regulations of protein functions and interactions, and so on.

Example of biopolymers having a sugar chain include proteoglycans in plant cell walls contributing to stabilization of cells; glycolipids influencing cell differentiation, proliferation, adhesion, migration, and so forth; glycoproteins associated with intercellular interaction and cell recognition; and the like. There have been gradually revealed mechanisms of how these polymeric sugar chains control biological reactions in a highly precise manner while mutually substituting, assisting, enhancing, regulating, or inhibiting their functions. Further, if involvements of such sugar chains in differentiation and proliferation of cells, cell adhesion, immunity, and carcinogenesis of cells are elucidated, a new development can be expected by closely relating this sugar chain engineering to medical science, cell engineering, or organ engineering (NPL 1).

In order to keep the quality of life (QOL) high through early detection of diseases, a biomarker is necessary which enables prevention of development of diseases and diagnosis of transition of diseases. From the analyses on gene-disrupted mice lacking a glycosyltransferase involved in sugar chain biosynthesis, it has been revealed that sugar chains are essential for maintenance of functions of various tissues and organs (NPLs 2, 3). Moreover, it has also been known that abnormal modifications of sugar chains may cause various diseases (NPL 4). Since the structures of sugar chains are significantly changed due to carcinogenesis of cells and various diseases, sugar chains are expected to be utilized as biomarkers to examine the transition of diseases.

MALDI-TOF MS is capable of easy and quick measurement without the need of complicated sample preparation, and thus recently considered to be one of powerful tools for biomarker searching (PTLs 1 to 3). However, one of the challenges in analyzing a sugar chain by MALDI-TOF MS is to improve the sensitivity of the analysis.

### CITATION LIST

### Patent Literature

[PTL 1] Japanese Unexamined Patent Application Publication No. 2009-142238

[PTL 2] Japanese Unexamined Patent Application Publication No. 2009-216609

[PTL 3] Japanese Unexamined Patent Application Publication No. 2009-229426

### Non Patent Literature

[NPL 1] Introduction to Glycobiology, Kagaku-Dojin Publishing Co., Inc., published on Nov. 1, 2005, first edition

[NPL 2] Ioffe E., Stanley P., Proc. Natl. Acad. Sci., 91, pp. 728-732 (1994)

[NPL 3] Metzler M., Gertz A., Sarker M., Schachter H., Schrader J. W., Marth J. D., EMBO J., 13, pp. 2056-2065 (1994)

[NPL 4] Powell L. D., Paneerselvam K., Vij R., Diaz S., Manzi A., Buist N., Freeze H., Varki A., J. Clin. Invest., 94, pp. 1901-1909 (1994)

### SUMMARY OF INVENTION

### Technical Problem

The present invention has been made in consideration of such circumstances. An object of the present invention is to provide a method for analyzing a sugar chain contained in a biological sample by a mass spectrometry method represented by MALDI-TOF MS with high sensitivity.

### Solution to Problem

The present inventors have earnestly studied in consideration of and to solve the following problem: since a sugar chain is a high-polarity neutral substance containing many hydroxyl groups, the ionization efficiency is low in conducting mass spectrometry, and accordingly the accuracy of the conducted mass spectrometry is low. As a result, the inventors have found out that it is possible to increase the ionization efficiency of a sample sugar chain by methylating hydroxyl groups of the sugar chain before MALDI-TOF MS measurement. This enables highly-sensitive quantitative and structural analyses on a sample sugar chain. This discovery has led to the completion of the present invention.

In other words, the present invention relates to a method for analyzing a sugar chain contained in a biological sample by a mass spectrometry method after methylating hydroxyl groups of the sugar chain. More specifically, the present invention provides the following inventions.
[1] A method for analyzing a sugar chain contained in a biological sample by a mass spectrometry method, comprising the steps of:

(a) releasing a sugar chain from a biological sample;

(b) capturing the released sugar chain onto a solid phase support;

(c) re-releasing the captured sugar chain;

(d) methylating a hydroxyl group of the re-released sugar chain; and

(e) analyzing the methylated sugar chain by a mass spectrometry method.
[2] A kit for performing the method according to [1], comprising a reagent for methylating a hydroxyl group of a sugar chain.

### Advantageous Effects of Invention

The present invention makes it possible to analyze a sugar chain contained in a biological sample by using MALDI-TOF MS with high sensitivity. Moreover, when a sugar chain having hydroxyl groups methylated is cleaved by acid hydrolysis or the like, a hydroxyl group is generated only at the cleaved site. Thus, the present invention also makes it possible to analyze the cleaved site and bonding pattern of the sugar chain.

### BRIEF DESCRIPTION OF DRAWING

FIG. 1 is a graph showing the result of MALDI-TOF MS analysis on N-Linked sugar chains of bovine serum IgG.

### DESCRIPTION OF EMBODIMENTS

(Step of Releasing Sugar Chain from Biological Sample)

As a sample containing sugar chains used in the present invention, it is possible to use biological samples such as, for example, whole blood, serum, plasma, urine, saliva, cell, tissue, virus, and plant tissue. It is also possible to use purified or unpurified glycoproteins. The sample maybe pretreated by a method such as degreasing, desalting, protein fractionation, or heat denaturation.

Sugar chains are released from a glycoprotein contained in the biological sample by using sugar chain-releasing means. As the means for releasing sugar chains, it is possible to employ a glycosidase treatment using N-glycosidase or O-glycosidase, hydrazine decomposition, β elimination under an alkali treatment, or the like. When an N-type sugar chain is analyzed, a method using N-glycosidase is preferable. Prior to the glycosidase treatment, a protease treatment may be performed using trypsin, chymotrypsin, or the like.

(Step of Capturing Released Sugar Chain onto Solid Phase Support)

A solution containing the sugar chains is brought into contact with a capturing support which specifically binds to a sugar chain, and thereby the sugar chains are captured on the capturing support.

Among biological substances, sugar chain is only a substance which has an aldehyde group. Specifically, sugar chain exists in equilibrium between the cyclic hemiacetal form and the non-cyclic aldehyde form under a condition such as in an aqueous solution. Biological substances other than sugar chains, such as proteins, nucleic acids, and lipids, contain no aldehyde group. Accordingly, it is possible to selectively capture only sugar chains by utilizing a capturing support which has a functional group specifically reacting with an aldehyde group to form a stable bond. Examples of a preferable functional group specifically reacting with an aldehyde group include an oxylamino group, a hydrazide group, an amino group, a semithiocarbazide group, and derivatives thereof. A hydrazide group or an oxylamino group is more preferable. Since an oxime bond formed by the reaction between an oxylamino group and an aldehyde group as well as a hydrazone bond formed by the reaction between a hydrazide group and an aldehyde group is easily cleaved by an acid treatment or the like, the sugar chain can be readily cleaved and separated from the support on which the sugar chain has been captured. Generally, an amino group is often used for capturing and retaining physiologically active substances. However, the binding strength of the bond (Schiff base) formed by the reaction between an amino group and an aldehyde group is weak, and accordingly a secondary treatment using a reducing agent or the like is necessary. Hence, an amino group is not preferable for capturing sugar chains.

As the support for capturing the sugar chains, polymer particles are preferably used. The polymer particles are preferably solid or gel particles having a functional group at least on a part of the surface, the functional group specifically reacting with an aldehyde group of a sugar chain. When the polymer particles are solid particles or gel particles, the sugar chains captured on the polymer particles can be readily recovered by means such as centrifugation, filtration, or the like. Meanwhile, the polymer particles can be used while filled in a column. The method using the polymer particles filled in a column is important particularly from the viewpoint of successive operations. The use of a filter plate (for example, MultiScreen Solvinert Filter Plate manufactured by Millipore Corporation) as a reaction vessel enables simultaneous treatment on multiple samples. The throughput of the sugar chain purification is greatly improved in comparison with conventional purification means, for example, using a column, represented by gel filtration.

The shape of the polymer particle is not particularly limited, but the shape is preferably spherical or similar shapes. In the case of spherical polymer particles, the average particle diameter is preferably 0.05 to 1000 μm, more preferably 0.05 to 200 μm, further preferably 0.1 to 200 μm, and most preferably 0.1 to 100 μm. If the average particle diameter is below the lower limit, it becomes necessary to apply a high pressure to compensate for poor passage of the solution through a column filled with the polymer particles thus used. In addition, it is difficult to recover the polymer particles by centrifugation or filtration. If the average particle diameter exceeds the upper limit, the contact area between the polymer particles and the sample solution is small, and the efficiency of capturing the sugar chains is decreased. In the present invention, “BlotGlyco®” (manufactured by Sumitomo Bakelite Co., Ltd., #BS-45603), which is hydrazide group-containing polymer particles, can be suitably used.

The pH of the reaction system for capturing the sugar chains with the polymer particles specifically capturing a sugar chain is preferably 2 to 9, more preferably 2 to 7, and further preferably 2 to 6. For the pH adjustment, various buffer solutions can be used. The temperature during the capturing of the sugar chain is preferably 4 to 90° C., more preferably 4 to 70° C., further preferably 30 to 80° C., and most preferably 40 to 80° C. The reaction time can be set as appropriate. The sample solution may be passed through a column filled with the polymer particles.

When the polymer particles are used, matters other than the sugar chains are non-specifically adsorbed to the support surface, and have to be removed by washing. As the washing liquid, water, a buffer solution, water or a buffer solution containing a surfactant, an organic solvent, and the like are preferably used in appropriate combination. A particularly preferable embodiment is a method including: washing sufficiently with water or a buffer solution containing a surfactant; then washing with an organic solvent; and lastly washing with water. By these washing, non-specifically adsorbed matters are removed from the surface of the polymer particles.

An excessive functional group on the support can be capped by utilizing, for example, acetic anhydride or the like.

(Step of Re-Releasing Captured Sugar Chain)

Next, the sugar chains bound to the polymer particle serving as the capturing support are re-released to obtain a purified sugar chain sample.

Description is given for a step of substituting the sugar chains bound to the polymer particles with another compound (hereinafter referred to as “compound A”). The compound A is preferably a labeling reagent. The substitution is carried out by adding an excessive amount of the compound A to the polymer particles on which the sugar chains are bound. Specifically, the sugar chain is cleaved and separated from the polymer particles, and simultaneously the compound A is added to the sugar chain (the sugar chain is “labeled” with the compound A). The amount of the compound A added excessively is preferably 1.5 times or more, more preferably 3 times or more, further preferably 5 times or more, and most preferably 10 times or more, of the amount of functional group specifically reacting with the sugar chain on the polymer particles. The pH of the reaction system is preferably 2 to 9, more preferably 2 to 7, and further preferably 2 to 6. For the pH adjustment, various buffer solutions can be used. The temperature of the reaction system is preferably 40 to 90° C., more preferably 40 to 80° C. Preferably, the solvent has been evaporated by the end of this step.

The compound A is preferably a compound having an aminooxy group or a hydrazide group. The most preferable compound is N-aminooxyacetyl-tryptophanyl (arginine methyl ester). The compound is commercially available as a re-releasing reagent appended to the above “BlotGlyco® for MALDI” (manufactured by Sumitomo Bakelite Co., Ltd., #BS-45603).

Hereinabove, the description has been given for the method to obtain a labeled sample by releasing the sugar chains captured on the sugar chain-capturing substance. Nevertheless, it is possible to obtain a non-labeled sample by the following method. The present invention also provides such a sample preparation method. This sample preparation method is characterized by treating polymer particles on which a sugar chain is bound under an acidic condition to dissociate a hydrazone bond, thereby releasing the sugar chain. The treatment under the acidic condition in this case is a treatment using a 0.01 to 20% acetic acid solution by volume, preferably a 0.1 to 5% acetic acid solution by volume. The treatment is performed at 40 to 80° C. for 5 to 60 minutes. Preferably, the solvent has been evaporated by the end of this step.

(Step of Methylating Hydroxyl Group of Re-Released Sugar Chain)

Next, hydroxyl groups of the re-released sugar chains are methylated. An example of the methylating reagent is iodomethane. The amount of the methylating reagent is preferably 10 to 200 μL, more preferably 10 to 100 μL. The methylating reagent is preferably a mixture solution with dimethyl sulfoxide (DMSO), more preferably a NaOH/DMSO slurry. Here, the NaOH/DMSO slurry is obtained by dispersing powdery solid NaOH in DMSO.

The methylating reagent solution is added to the polymer particles after the above-described step of re-releasing the sugar chain. The resultant is left standing at a predetermined temperature for a predetermined period. Thereby, the sugar chains are methylated. The methylating step is normally performed at 10 to 60° C., preferably 10 to 40° C., for 10 minutes.

The methylating reaction can be stopped by adding pure water. The methylated sugar chains can be recovered from the reaction solution by, for example, chloroform extraction. For example, the reaction solution of the methylating reaction is added to chloroform. Then, the chloroform layer is collected, and washed with pure water. Subsequently, the chloroform solution is evaporated for use in mass spectrometry described below.

As described above, according to the method of the present invention, it is not necessary to separate the re-released sugar chains from the polymer particles, and the methylation can be performed while the sugar chains are mixed with the polymer particles. Meanwhile, since the methylating reaction is a reaction that should be carried out in the absence of water, normally the sugar chain solution is dried by a method such as lyophilization for use in the reaction. In the method of the present invention, such a drying step is not necessary because the solvent is evaporated after the step of re-releasing the sugar chain. Thus, the present invent ion provide s a method of purifying and methylating sugar chains by simple operations.

(Step of Analyzing Methylated Sugar Chain by Mass Spectrometry Method)

The obtained sugar chains can be analyzed by a mass spectrometry method represented by MALDI-TOF MS. The mass spectrum obtained by MALDI-TOF MS measurement can be analyzed using analysis software or the like. Peaks of the sample sugar chains are detected by reading the mass-to-charge ratio (m/z value). The quantification and the structural analysis of a sugar chain can be conducted based on the mass-to-charge ratio and the peak intensity (any index such as peak height or peak area). In the analysis of a sugar chain, various database (for example, GlycoMod, Glycosuite, and the like) can be utilized.

(Kit)

The present invention provides a kit for performing the method of the present invention, comprising a reagent for methylating a hydroxyl group of a sugar chain. The kit of the present invention may comprise, other than the methylating reagent, for example, (1) a support (such as beads) for capturing (purifying) a sugar chain, (2) reagents for labeling and/or re-releasing the sugar chain captured on the support, (3) a support (such as a column) for removing the reagent (2) remaining in an excessive amount in a reaction solution, (4) a reaction tube, and (5) an instruction.

### EXAMPLES

Hereinafter, the present invention will be described more specifically based on Examples. However, the present invention is not limited to the following Examples.

### Example 1

### Releasing Sugar Chains from Glycoprotein

Into a tube, 1 mg of bovine serum immunoglobulin G (IgG, manufactured by Sigma-Aldrich Corporation, #I5506) was put and completely dissolved by adding a 1 M aqueous solution of ammonium bicarbonate (5 μL), pure water (50 μL), and a 120 mM aqueous solution of dithiothreitol (5 μL) thereto. Then, the mixture was incubated at 60° C. for minutes. Next, a 123 mM aqueous solution of iodoacetamide (10 μL) was added. The tube was shielded from light and left standing at room temperature for 1 hour. Subsequently, 400 units of trypsin was added thereto, and the resultant was left standing at 37° C. for 1 hour. By heating at 95° C. for 5 minutes, trypsin was inactivated. Thereafter, 5 units of N-glycosidase F (manufactured by Roche Diagnostics K.K., #11365193001) was added, and the resultant was left standing at 37° C. for 16 hours. Thereby, sugar chains were released.

### Example 2

### Supporting Sugar Chains onto Polymer Particles

To 5 mg of polymer particles (manufactured by Sumitomo Bakelite Co., Ltd., BS-45603), 20 μL (corresponding to 100 μg of IgG) of thus obtained solution of the released sugar chains was added, and 180 μL of acetonitrile containing 2% acetic acid was further added. Then, the mixture was allowed to react at 80° C. for 1 hour, and solidified by drying. The polymer particles were washed with a 2 M guanidine hydrochloride solution, water, methanol, and a 1% triethylamine solution. Subsequently, a 10% acetic anhydride/methanol solution was added thereto, and the mixture was allowed to react at room temperature for 30 minutes, thereby capping the hydrazide groups. After the capping, the polymer particles were washed with methanol and water.

### Example 3

### Re-Releasing Sugar Chains

To the above prepared polymer particles supporting the sugar chains, 20 μL of pure water and 180 μL of acetonitrile containing 2% acetic acid were added. Then, the mixture was allowed to react at 80° C. for 1 hour, and solidified by drying. Thereby, the sugar chains were released from the polymer particles.

### Example 4

### Preparing Methylating Reagent Solution

Approximately 1 g of granular sodium hydroxide was placed in a mortar and quickly ground with a pestle. To this, 1 mL of DMSO was added and mixed. Thereby, a slurry solution was prepared. With 100 μL of this solution, 100 μL of iodomethane (manufactured by Wako Pure Chemical Industries, Ltd., #131-02661) was mixed. Thus, a methylating reagent solution was prepared.

### Example 5

### Methylating Sugar Chain

After the sugar chains were re-released, 100 μL of the methylating reagent solution was added to the polymer particles. The mixture was left standing at 25° C. for 10 minutes and allowed to react. Then, the reaction was stopped by adding 200 μL of pure water was added thereto. The polymer particles were separated from the solution by filtration, and the solution was collected. The solution was transferred into a sample tube, and 400 μL of chloroform was added thereto, followed by stirring for 1 minutes. Thereby, the methylated sugar chains were recovered in the chloroform layer. The sample tube was left standing to separate the chloroform layer from the aqueous layer. Thereafter, the chloroform layer was collected into another sample tube. To this, 800 μL of pure water was added, followed by stirring for 1 minute. Thereby, water-soluble impurities contained in the chloroform layer were removed. The sample tube was left standing to separate the chloroform layer from the aqueous layer. The chloroform layer was collected into another sample tube, and dried with a centrifugal drier. Thus, the methylated sugar chains were obtained.

### Example 6

### MALDI-TOF MS Measurement

The methylated sugar chains thus obtained were dissolved in 100 μL of methanol. This solution was diluted 10-fold with a matrix solution (aqueous solution of 10 mg/mL 2,5-dihydroxybenzoic acid). Of the resulting solution, 1 μL was spotted on a sample stage, followed by drying and crystallization. This was analyzed with a matrix-assisted laser ionization time-of-flight mass spectrometer (MALDI-TOF MS) (manufactured by Bruker Daltonics Inc., ‘autoflex III’). The measurement was made in positive ion detection mode and reflectron mode.

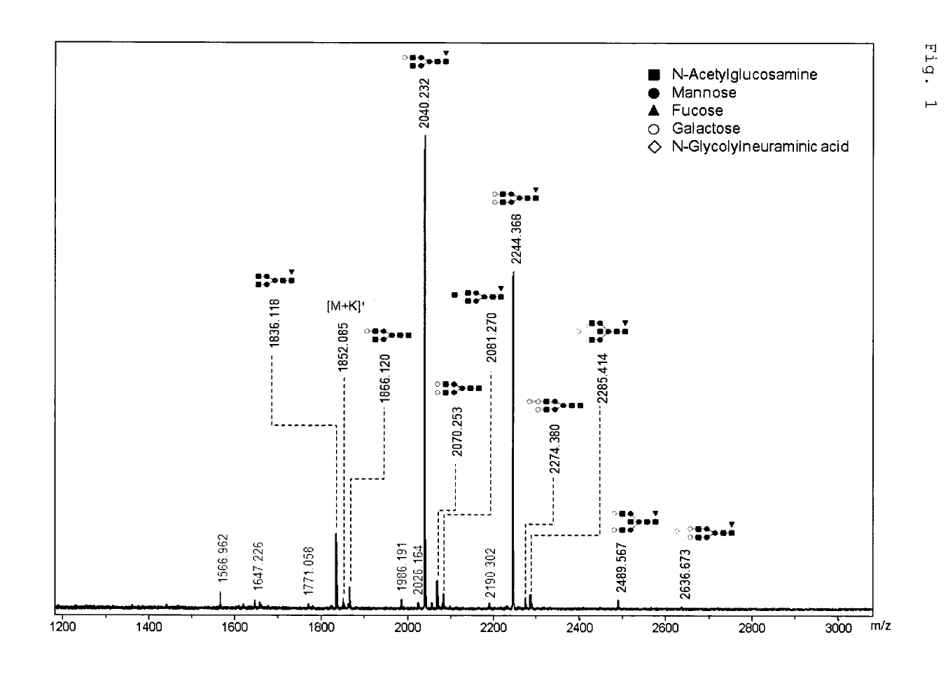

The obtained mass spectrum is shown in (FIG. 1). The sugar chain composition was estimated from the mass number-to-charge ratio (m/z value) obtained from the spectrum. The sugar chain composition was estimated using GlycoMod tool (http://www.expasy.org/tools/glycomod/) and GlycoSuite database (http://glycosuitedb.expasy.org/glycosuite/glycodb). The schematic diagrams of the estimated sugar chains are shown in the mass spectrum of FIG. 1.

As described above, it was demonstrated that it was possible to conduct quantitative and structural analyses on a sample sugar chain with high sensitivity by MALDI-TOF MS measurement after methylating hydroxyl groups of the sample sugar chain.

### INDUSTRIAL APPLICABILITY

The use of the method for analyzing a sugar chain of the present invention enables highly-sensitive quantitative analysis (quantitative profiling) and structural analysis on a sugar chain of a glycoprotein contained in a biological sample such as, for example, serum or tissue of a patient with a disease. The method is applicable to search for a sugar chain as a disease marker and to the medical field such as kinetic study on a sugar chain upon drug administration.

1. A method for analyzing a sugar chain contained in a biological sample by a mass spectrometry method, comprising the steps of:
(a) releasing a sugar chain from a biological sample;
(b) capturing the released sugar chain onto a solid phase support;
(c) re-releasing the captured sugar chain;
(d) methylating a hydroxyl group of the re-released sugar chain; and
(e) analyzing the methylated sugar chain by a mass spectrometry method.

2. A kit for performing the method according to claim 1, comprising a reagent for methylating a hydroxyl group of a sugar chain.
